(19) United States
(12) Patent Application Publication
(10) Pub. No.: US 2004/0197288 A1
(43) Pub. Date: Oct. 7, 2004

(54) Polymers derived from polysaccharides comprising one or more oxime or amine functions, and uses thereof

(76) Inventors: Etienne Fleury, Irigny (FR); Alain Domard, Lyon (FR)

Correspondence Address: Jean Louis Seugnet, Intellectual Property Department, Rhodia Inc CN 7500, 259 Prospect Plains Road, Cranbury, NJ, 08512-7500, US

(21) Appl. No.: 10/481,931

(22) PCT Filed: Jul. 1, 2002
(86) PCT No.: PCT/FR02/02288

(30) Foreign Application Priority Data
Jun. 29, 2001 (FR) 01/08878

(51) Int. Cl. A61K 7/06; A61K 7/11; C08G 63/48; C08G 63/91

(52) U.S. Cl. 424/70.13; 525/54.2

(57) ABSTRACT

The invention concerns a polymer derived from a polysaccharide copolymer consisting of a main chain comprising several similar or different anhydrohexose units, and branches comprising at least a neutral or anionic anhydropentose and/or anhydrohexose unit; said polymer derivative comprising one or several units bearing an oxime function at least on position C2. Furthermore, the polymer is obtainable by carrying out the following steps: a) contacting a polysaccharide with an aqueous solution comprising at least an oxidising agent for oxidising at least the hydroxyl radical borne by the carbon C2 of one or several units, into a ketone function; b) contacting the resulting polymer with hydroxylamine or a derivative to transform the ketone function into an oxime function. The invention also concerns a polymer obtainable by carrying out a step c) which consists in contacting the polymer having at least an oxime function at least on position C2 of said unit, with an agent reducing the oxime function. The resulting polymer bears an amine function at least on position C2. The invention further concerns the uses of said polymers.

The present invention relates to a polymer which is derived from a polysaccharide which carries lateral sugars and carries at least an oxime function in the C2 position. It also relates to a polymer which comprises at least an amine function in the C2 position, with this polymer being obtained from the preceding polymer. Finally, the invention relates to the use of these polymers.

Most polysaccharides which are encountered comprise chains of various sugars which are linked to each other and which, in addition to the primary alcohol functions or, possibly, aldehyde or ketone functions, mainly exhibit secondary alcohol functions.

One of the rare polysaccharides to differ in this regard is chitosan. This polysaccharide is obtained by deacetylating chitin, which is a structural polymer in the exoskeletons of arthropods, the endoskeletons of cephalopods or else the cell walls of some fungi or algae. It consists of repeated D-glucosamine units which are linked β-(1->4), containing up to 40% N-acetylglucosamine residues. In the case of the lowest contents of acetylated residues, the intrinsic pK of the amine function in protonated form is very low (approximately 6.5) as compared with the other polyamines; chitosan possesses, in particular, metal-chelating capacities which are not possessed by the other polysaccharides.

However, one of the major drawbacks of chitosan is the cost of this product.

The high cost of chitosan is due to the fact that chitin, which is the product from which it is derived, originates from the carapaces of marine animals such as crabs, krill, the squid endoskele-ton, etc. Steps of preliminary treatment therefore have to be carried out in order to extract the chitin. In addition, the volumes of chitin which are available are limited simply due to the origin of this product.

A further constraint is that of the chemical treatments which are required for obtaining the chitosan. Thus, the deacetylation is most frequently effected using 40-50% sodium hydroxide solution. Furthermore, these chemical treatments can prove to be polluting and therefore make it necessary to implement processes for treating the effluents, thereby increasing still further the cost of the final product.

The object of the present invention is therefore that of proposing modified polysaccharides which possess application properties which are as attractive, if not more attractive, than those observed with chitosan, without suffering from the drawbacks of the latter.

These aims, and others, are achieved by the present invention, which therefore relates, in the first place, to a polymer which is derived from a copolymeric polysaccharide which is formed from a main chain which comprises similar or different anhydrohexose units and branches which comprise at least one neutral or anionic anhydropentose and/or anhydrohexose unit; said derived polymer comprises one or more units which carry(ies) an oxime function at least in the C2 position and can be obtained by implementing the following steps:

a) bringing a polysaccharide into contact with an aqueous solution which comprises at least one oxidizing agent which enables at least the hydroxyl radical carried by the C2 carbon of one or more units to be oxidized to a ketone function;

b) bringing the resulting polymer into contact with hydroxylamine, or a derivative, in order to transform the ketone function into an oxime function.

In the second place, the present invention relates to a polymer which is derived from a copolymeric polysaccharide which is formed from a main chain which comprises similar or different anhydrohexose units and branches which comprise at least one neutral or anionic anhydropentose and/or anhydrohexose unit; said polymer comprises one or more units which carry(ies) an amine function at least in the C2 position and can be obtained by implementing a step c) which consists in bringing the polymer possessing one or more units carrying an oxime function at least in the C2 position into contact with an agent which reduces the oxime function.

The invention also relates to the use of the polymer exhibiting the oxime functions as an agent for complexing cationic species, more especially cationic species which comprise at least one metal.

Finally, the invention relates to the use of the polymers exhibiting amine functions in biomedicine, in surface treatment, in particular the treatment of textile surfaces, and in the papermaking industry.

Thus, it has been observed that the polymers according to the invention, more specifically those possessing the oxime functions, exhibit an outstanding ability to complex, in particular, metal cations, such as iron, chromium, mercury, silver, cadmium, uranium, etc. It was also noted that, in the case of the polymer according to the invention, it was possible to observe this complexation phenomenon in a pH range which is wider than that in which this phenomenon is observed in the case of chitosan. This is an undoubted advantage since it increases the range over which said polymer is effective and can be used.

As far as the polymers exhibiting the amine functions are concerned, it was found that they were able to react with negatively charged surfaces and confer various properties on these surfaces. Consequently, these polymers are particularly appropriate for being used, among other applications, as agents for protecting surfaces such as textile materials.

Furthermore, these polymers can be obtained from polysaccharides which are available in substantial and renewable quantities. This is because the polysaccharides which can be used for preparing the polymers according to the invention are derived from plants, such as cyanopsis tetragonoloba, and carob seed. Furthermore, the polysaccharides are extracted very simply from these sources and do not require any specific chemical transformation.

However, other advantages and features of the invention will appear more clearly from reading the description and examples which follow.

The polymers according to the invention are therefore derivatives of polysaccharides which are formed from a main chain which comprises similar or different anhydrohexose units and branches which comprise at least one neutral or anionic anhydropentose and/or anhydrohexose unit.

It is to be noted that, according to the invention, the term copolymeric polysaccharide signifies that the polymer is not selected from those in which all the constituent units are identical. In that which follows, and unless otherwise indicated, the term “polysaccharide” will be used instead of copolymeric polysaccharide.

The (similar or different) hexose units of the main chain of the native skeleton of the polysaccharide can, in particular, be units which are selected from D-glucose, D- or L-galactose, D-mannose, D- or L-fucose, L-rhamnose, etc.

The (similar or different) neutral or anionic pentose and/or hexose units of the branches of the native skeleton of the polysaccharide can, more specifically, be units which are selected from D-xylose, L- or D-arabinose, D-glucose, D- or L-galactose, D-mannose, D- or L-fucose, L-rhamnose, D-glucuronic acid, D-galacturonic acid and D-mannuronic acid, among others.

Furthermore, the polysaccharides from which the polymers according to the invention are obtained can be used in the native state or else after having undergone one or more depolymerization processes.

The galactomannans, the galactoglucomannans, the xyloglucans, the succinoglycans, the rhamsans and the welan gums, inter alia, may be mentioned as examples of native polysaccharide skeletons.

The native skeleton of the polysaccharide from which the polymer according to the invention is derived is preferably a galactomannan.

The galactomannans are macromolecules which are composed of a main chain of D-mannopyranose units, linked in the β(1-4) position, which is substituted by D-galactopyranose units in the α(1-6) position.

They are extracted from the albumen of leguminous seeds, of which they constitute the reserve carbohydrate. Preferred galactomannans which may be mentioned are guar gum, which is derived from guar seeds (Cyanopsis tetragonoloba), carob gum, with this being extracted from the seeds of the carob tree (Ceratonia siliqua), tara gum and cassia gum.

Quite preferably, the native skeleton is a guar gum. More specifically, the guar gums exhibit a mannose/galactose ratio of 2.

The weight average molar mass of the polysaccharides which can be used for obtaining the polymers according to the invention can vary over a wide range. However, said polysaccharides advantageously exhibit a weight average molar mass of between 10⁴ and 3.10⁶ g/mol (determined by size exclusion chromatography).

The polysaccharides can be used in the form of a powder or of particles of a few millimeters in size. It is to be noted that, in the case of galactomannans such as, in particular, guar, said particles are termed splits and consist of the seed cotyledons, which have been separated from the central germ and the envelope. These splits can also contain water. The content of water depends to some degree on the humidity of the ambient air. However, by way of illustration, the content of water is generally less than or equal to 10% by dry weight.

The polymers according to the invention therefore comprise one or more units which are carrying an oxime function at least in the C2 position in the unit. The other positions in the unit, in addition to the carbon in the C2 position, which are capable of carrying such a function are, possibly, the C3 carbon atoms and, possibly, the C4 carbon atoms. It is to be noted that, depending on the nature of the unit and its position in the polymer chain (main chain or branch), it may not be possible to oxidize the carbon in this position. In addition, it is pointed out that the polymers according to the invention may comprise units which do not carry any oxime function. Furthermore, the polymer according to the invention can, where appropriate, carry a carboxylic acid function (—COOH) in the C6 position in one or more units.

When the polymer according to the invention is composed of different types of oxidized carbon atoms (C2 and C3, C4 or C6), said atoms may or may not be located in the same unit.

It is recalled that the C2 carbon is located in the α position to the anomeric carbon.

According to one particularly advantageous embodiment of the present invention, the polymers are such that the majority (more than 50% by number) of the oxime functions are carried by the carbon atoms in the C2 position.

A preferred variant of the invention consists of a polymer which is obtained from native or non-native (more specifically depolymerized) guar and which carries an oxime function which is mainly in the C2 position, with the majority of the substituted units being those which constitute the branches of the guar.

The first step in the process which can be used for preparing the polymers according to the invention will be described first of all. Step a) consists in bringing the native or non-native polysaccharide into contact with an aqueous solution which comprises at least one oxidizing agent which enables at least the hydroxyl radical carried by the C2 carbon in one or more units to be oxidized to a ketone function.

A first embodiment of step a) consists in using the polysaccharide in the form of an aqueous solution.

According to this embodiment, step a) is carried out in a homogeneous phase.

A second embodiment of step a) consists in using the polysaccharide in the form of a powder or of particles in the presence of an organic compound which is selected from the solvents which do not dissolve the polysaccharide (nonsolvents). Under these conditions, the process according to the invention is implemented in a heterogeneous form.

Said organic compound is selected from the compounds which are inert under the conditions of the reaction. Furthermore, said compounds are preferably selected from the compounds which are at least partially miscible with water. Examples of these compounds which may be mentioned are, inter alia, hindered or unhindered alcohols, such as, very particularly, methanol, ethanol, isopropanol or tert-butanol; and ketones, such as acetone.

Within the context of the second embodiment of step a), the reaction can be carried out in the presence of water. However, the quantity of water which is involved during this step is such that the polysaccharide remains in the form of powder or particles which is/are dispersed in the reaction mixture. Thus, if step a) takes place in the presence of water, the content of the latter preferably does not in general exceed 30% by weight of the reaction mixture.

With regard to the nature of the oxidizing agent, the latter is advantageously selected from bromine, the periodate of an alkali metal such as sodium, or derivatives of 2,2,6,6-tetramethyl-1-piperidyloxy (TEMPO), more specifically combined with the hypochlorite of an alkali metal, such as sodium, in the presence of a bromide of an alkali metal (preferably sodium). The oxidizing agent employed is preferably bromine.

It is to be noted that the oxidizing agent is used in the form of an aqueous solution. The quantity of water supplied with the oxidizing agent is such that the reaction is still carried out in heterogeneous phase. Thus, the content of water supplied together with the oxidizing agent is preferably such that the maximum content of water in the reaction mixture is less than or equal to 30% by weight of the reaction mixture.

In certain cases, in particular that in which step a) is carried out using periodate, it can be advantageous to recycle this oxidizing agent in the usual manner.

Furthermore, the molar ratio of the oxidizing agent to the functions to be oxidized is more specifically less than 6, more particularly less than 4 and preferably between 1 and 2.5. It is to be noted that some oxidizing agents can be employed in catalytic quantity.

According to one advantageous embodiment, step a) is carried out by adding the oxidizing agent to the polysaccharide.

In addition, a particularly appropriate variant of the invention consists in maintaining the pH during step a). The pH of the aqueous solution is preferably maintained at a value between 6 and 8, very advantageously at a pH between 6.5 and 7.5.

The pH can be maintained by adding a base. It is to be noted that the base can either be added directly to the reaction mixture or added to the solution of the oxidizing agent.

The temperature at which step a) is carried out is preferably between 0 and 70° C., advantageously between 10 and 30° C.

The skilled person can set the duration of step a) without difficulty using standard analytical methods (¹³C NMR, infrared). As a simple illustration, and in the case of reaction conducted discontinuously and in homogeneous phase, the duration of step a) is less than 60 minutes, more specifically less than or equal to 30 minutes, and preferably between 10 and 25 minutes. It is pointed out that this length of time does not include that of introducing the oxidizing agent.

Attention is drawn to the fact that the polymer derived from step a) can exhibit a carboxylic function on one or more of the repeated units of the initial polysaccharide. Such a function can be obtained by oxidizing the primary alcohol function if it is present in the unit under consideration.

Furthermore, having been selectively oxidized during step a), the C2 carbon atom and, where appropriate, the C3 or C4 carbon atoms can be present in two different forms, with the one being in equilibrium with the other. Thus, the ketone form can be present in equilibrium with the hydrated ketone form (^{HO}>C<^{OH}).

At the conclusion of step a), which has just been described, the resulting polymer is such that it exhibits an average degree of substitution of the secondary hydroxyl functions, more specifically carried by the C2 carbon and, where appropriate, by the C3 or C4 carbon atoms, of between 0.01 and 2, preferably of between 0.1 and 1.

It is pointed out that the average degree of substitution is calculated from carbon 13 NMR spectra and, more specifically, from the integrals of the unresolved peaks which are characteristic of the functions which are present in the resulting polysaccharide.

Its value is given by the following calculation:

$$DS = \frac{( I \times 100 )}{( {Ic}_{total} )} \times \frac{{DS}_{max}}{( 100 C α / C β )}$$

in which:

I denotes the integral of the unresolved peak under consideration

Icₜₒₜₐₗ denotes the integral of all the unresolved peaks

DSₘₐₓ denotes the maximum degree of substitution; it is

2 in the case of the ketone functions;

Cα denotes the number of carbons which can be modified per repeated unit (example: galactose; mannose: Ca=4);

Cβ denotes the average total number of carbons per repeated unit (example: galactose; mannose: Cp=6).

At the conclusion of this step a), the resulting polymer is preferably separated from the reaction medium.

When the reaction has taken place in homogeneous medium, the separation can be effected by adding a nonsolvent of the resulting polymer to the reaction medium. The nonsolvents mentioned within the context of the variant relating to the reaction in heterogeneous phase are suitable and it is possible, therefore, to refer to them.

The polymer is then separated by filtration or centrifugation.

When the reaction is carried out in heterogeneous medium, this separation is effected by simple filtration or centrifugation.

As has previously been mentioned, step b) consists in bringing the resulting polymer into contact with hydroxylamine, or a derivative, in order to transform the ketone function into an oxime function.

It is to be noted that, if a hydroxylamine derivative is used, it is selected from hydroxylamine sulfate and hydroxylamine chloride.

When step b) is carried out in the presence of a hydroxylamine derivative it is then preferable to carry out said step while maintaining the pH between 6 and 9.5. This can be effected, in particular, by adding a base during the course of this step.

According to an advantageous embodiment of this step b), the molar ratio of the hydroxylamine or derivative to the ketone functions to be transformed is between 1 and 10, preferably between 1 and 6.

In general, step b) is carried out using an aqueous solution of the hydroxylamine or hydroxylamine derivative.

Advantageously, the hydroxylamine or its derivative is employed in this step in the form of an aqueous solution in which the concentration of the hydroxylamine or derivative is between 20 and 60% by weight.

The temperature at which step d) is carried out is more specifically between 0 and 70° C., preferably between 10 and 30° C.

At the conclusion of this step b), the resulting polymer exhibits an average degree of substitution of the ketone functions of less than 2, preferably of between 0.01 and 2 excluded.

The average degree of substitution is once again determined from carbon 13 NMR spectra and, more specifically from the integrals of the unresolved peaks which are characteristic of the functions which are present in the resulting polysaccharide.

Its value is given by the following calculation:

$$DS = \frac{( I \times 100 )}{( {IC}_{total} )} \times \frac{{DS}_{max}}{( 100 C α / C β )}$$

in which:

I denotes the integral of the unresolved peak under consideration

ICₜₒₜₐₗ denotes the integral of all the unresolved peaks

DSₘₐₓ denotes the maximum degree of transformation; it is 2 in the case of the oxime functions;

Cα denotes the number of carbon atoms which can be modified per repeated unit (example: galactose; mannose: Cα=4);

Cβ denotes the average total number of carbons per repeated unit (example: galactose; mannose: Cβ=6).

Once again, it can be preferable to separate the resulting polymer from the reaction medium. This procedure can, in particular, take place in the same way as in the case of step a).

The present invention also relates to a polymer which is derived from a polysaccharide which is formed from a main chain comprising similar or different anhydrohexose units and from branches comprising at least one neutral or anionic anhydropentose and/or anhydrohexose unit; said polymer comprises one or more units which carry an amine function at least in the C2 position and can be obtained by implementing a step c) which consists in bringing the polymer possessing one or more units carrying an oxime function at least in the C2 position into contact with an agent which reduces the oxime function.

The polymers according to the invention therefore comprise one or more units which carry an amine function in the C2 position in the unit, and, where appropriate, on the C3 carbon atoms and, where appropriate, on the C4 carbon atoms. In addition, it is pointed out that the polymers according to the invention can comprise units which do not carry any oxime function. Furthermore, the polymer according to the invention can, where appropriate, carry a carboxylic acid function (—COOH) in the C6 position in one or more units.

Finally, when the polymer according to the invention comprises different types of carbon atom carrying an oxime function (C2 and C3 or C4), said atoms may or may not be located in the same unit.

The reaction which is brought into play for obtaining the polymer comprising one or more amine functions can take place by using, as the agent for reducing the oxime function to an amine function, an agent which is selected from lithium hydride and aluminum hydride; and boron compounds such as BH₃, NaBH₄, NaBH₃CN and NaBH₂S₃, which may or may not be combined with a Lewis acid. Lewis acids which can be used, preferably in combination with borohydride or cyanoborohydride, and which may be mentioned are molybdenum oxide, nickel chloride, titanium chloride and titanium oxide.

This type of agent is customarily, and advantageously, used in the presence of water, with the exception of lithium aluminum hydride which is preferably used in the presence of a solvent of the tetrahydrofuran type.

The temperature can vary over a wide range. As an indication, it is between 10° C. and the temperature at which the medium refluxes.

It is also possible to use hydrogen in the presence of a catalyst of the palladium on carbon type, where appropriate in the presence of hydrochloric acid, or of the platinum oxide type or Raney nickel type.

The pH at which the reaction is carried out can vary over a relatively extended range, depending on the nature of the reducing agent which is selected. Thus, as an example, and in the specific case of a reduction carried out using a borohydride, the pH is advantageously between 3 and 10.

Finally, this step c) is preferably carried out under an inert atmosphere. Thus, nitrogen or rare gases can be used in the appropriate manner.

The invention therefore relates, in the third place, to the use of the polymer comprising the oxime functions as an agent for complexing cationic species, preferably metallic cationic species. Cationic species which are capable of being complexed and which may be mentioned, in particular, are Cu²⁺, Au³⁺, V²⁺, V³⁺, V⁴⁺, V⁵⁺, U²⁺, U⁴⁺, Fe²⁺, Fe³⁺, Ru³⁺, Rh³⁺, Pd²⁺, Pd⁴⁺, Pt²⁺, Pt⁴⁺, Pu³⁺, Pu⁴⁺, Ir³⁺, Ir⁴⁺, Os³⁺, Os⁴⁺, Zn²⁺, Cd²⁺, Cr²⁺, Cr³⁺, Th⁴⁺, Co²⁺, Co³⁺, Ni²⁺, Ni³⁺, Ag⁺, Sb³⁺, Sn²⁺, Sn⁴⁺, Mn²⁺, Mn³⁺, Hg²⁺, Bi³⁺, Pb²⁺, Pb⁴⁺.

More specifically, said polymers can be used for extracting metals.

The polymers according to the invention, more specifically those possessing oxime functions, can also be used in the papermaking industry, in particular as a retention agent which may or may not be mixed with a multivalent metal, such as aluminum.

The invention finally relates to the use of the polymers possessing the amine functions in biomedicine or in surface treatment, in particular of the treatment of textile surfaces.

Examples which are specific, but which do not limit the invention, will now be presented.

### EXAMPLES

In the examples which follow:

1/ the mean molar masses by weight of the polymers arrying ketone or oxime functions are determined as ollows:

The measurement is carried out in: Millipore 18 MQ water, 30% MeOH, 0.1 M LiCl, pH 9-10 (2/10000 parts of NH₄OH)

The features of the equipment are as follows:

Chromatographic columns: 1 Shodex SB806HQ 30 cm, 5 μm column+1 Asahi GFA30 60 cm, 5 μm column.

Injection pump: Wisp 717+Waters 515 pump

Detector: Waters 410 R1 refractometer, sensitivity 8, Wyatt MALLS light scattering, He 633 nm laser

Flow rate: 0.5 ml/mn

The injected solution (200 μl) contains 1/1000 by weight of polysaccharide.

The weight average molecular mass is established directly without calibration using the light scattering values extrapolated to zero angle; these values are proportional to C×M×(dn/dc)2.

C corresponds to the concentration of polysaccharide

M corresponds to the weight average molecular mass

n corresponds to the refractive index of the solution

c corresponds to the concentration of polysaccharide

the ratio dn/dc is in this present case equal to 0.15.

2/nitrogen is determined using a Carlo Erba EA1108 analyzer, which analyzes C, H, N and S simultaneously in organic substances using the classical methods of Dumas and Pregl.

The analyzer comprises 2 essential parts having very distinct functions:

1. A reactor, which consists of a quartz column having tiered filling. This is the site of the successive transformations of the samples (combustion, oxidation and reduction) in order to extract the sought-after elements C, H, N and S in the form N₂, CO₂, H₂O and SO₂.

2. An analytical cell, which consists of a gas chromatography column and catharometric detection.

The processing of the data is managed entirely by a microcomputer which is equipped with the Eager 200 software supplied by the manufacturer Carlo Erba.

With the content of nitrogen being expressed in percentage by weight, the amine function DS is calculated using the following formula: DS=162.Y/(1400+15Y), where Y is the percentage of nitrogen by weight.

This formula is obtained bearing in mind that the polymer has the following empirical formula:

C₆H_{(10+DS)}O_{(5-DS)}N_{(DS)}.

### Example 1

### Synthesizing a Polymer which is Derived from Guar and Carries Ketone Functions

54 grams of guar (weight average molar mass: 50000 g/mol—Meyprogat® 7, marketed by Rhodia) are dissolved in 840 ml of water.

In addition, a solution of bromine is prepared by adding 26 ml of bromine to 250 ml of water and then neutralizing by adding sodium hydroxide solution (2N) in order to obtain a stable pH of 7.6.

The bromine solution which is thus obtained (2 equivalents per anhydropyrannose motif) is then added dropwise to the polymer.

While the bromine is being added, and until the pH no longer changes, sodium hydroxide solution (2N) is added so as to maintain the pH at approximately 7.

Wheh the pH has stabilized, the reaction mixture is poured into ethanol so as to precipitate the polymer which has been obtained. The polymer is then filtered on a no. 4 frit.

At the conclusion of this step, the polymer possesses ketone functions predominantly at positions C2 and C3.

For the analysis, the product is dried by lyophilization.

The ¹³C NMR spectrum is analyzed.

Conditions: 400 MHz

Solvent: D₂O

Temperature 70° C.

Accumulation time: approximately 48 hours.

Results:

Comparison of the spectrum of the starting polymer and of that derived from the reaction shows the appearance of three new unresolved peaks which are characteristic of the ketone, hydrated ketone and carboxylic acid functions.

In addition, the degree of ketone substitution, as calculated from the ¹³C NMR spectrum, is 0.53.

The weight average molar mass is 6500 g/mol.

### Example 2

### Synthesizing the Polymer Comprising the Oxime Functions

40 g of the product in the solid and dry state, as previously obtained, are dissolved in 700 ml of water. The pH of the solution is approximately 7.

76 ml of a 50% by weight solution of hydroxylamine in water are added to the solution.

After addition, the pH is 9.4.

The whole is left to stir for 18 hours. The pH is 9.1.

At the conclusion of this step, the product is separated off by precipitation in ethanol and filtration on a no. 4 frit.

For the analysis, the product is dried by lyophilization.

Comparison of the 13C NMR spectra (ambient T°, 4 hours) of the polymers obtained during the oxidation step and after the oximation step shows the appearance of an unresolved peak which is characteristic of the oxime function.

Elemental analysis of the polymer (determining the nitrogen by thermally degrading the compound and then using gas chromatography to analyze the emitted gases) indicates that the transformation of the ketone functions is almost quantitative.

The calculated degree of substitution is 0.46.

The weight average molar mass is 5100 g/mol.

### Example 3

### Synthesizing the Amine Derivative

8 ml of a 13% solution of TiCl₃ in 20% HCl, and 10 ml of water, are initially introduced into a stirred glass reactor.

2 g of NaOH which has been previously dissolved in 10 ml of water are then added. A violet precipitate is then formed.

10 ml of an aqueous solution containing 500 mg of NaBH₄ are then added to this mixture and, after stirring for 15 minutes, 1 g of guar oxime from the previous example, dissolved in 15 ml of water, is introduced.

The medium becomes white and very viscous; it is left to stir under nitrogen for 48 hours.

At the end of this period, the pH of the medium is 8.5.

The solid phase of the reaction medium is separated from the liquid phase by filtration and the modified guar, which is located in the liquid, is recovered by means of precipitation in a nonsolvent (ethanol) and filtration.

The polymer is analyzed by ¹³C NMR.

Under these conditions, it is seen that the unresolved peak of the oxime at 154 ppm has disappeared.

In parallel, peaks appear at about 54 ppm, corresponding to carbon atoms which are carrying an amine function.

The ¹H NMR confirms this result since there are characteristic signals of the proton in the α position of an amine function between 1 and 2.5 ppm.

Finally, elemental analysis enables the nitrogen content to be measured. It is 4.1 as compared with 4.2 in the case of the oximated product.

### Example 4

### Complexation Properties

Complexation of the Oxime-Modified Guar in the Presence of Copper:

The assays were carried out, at a constant pH, by adding varying quantities of copper perchlorate to a solution of oximated guar.

The concentration of the polymer is 16.8 g/l and the pH is adjusted by adding hydrochloric acid.

The solution is then left stirring at ambient temperature for 24 hours.

The appearance of a green color and the formation of a green precipitate are observed.

After 24 hours, the reaction medium is centrifuged.

The supernatant is filtered through a nylon filter (0.45 μm) and the filtrate is then analyzed by potentiometry. The potentiometry determines the free CU⁺⁺, and the complexed CU⁺⁺ is therefore obtained as the difference from the quantity which was introduced (see table 1).

It can be seen that the complexation ratio is close to the theoretical value, in particular at pH 5.

Furthermore, there is a tendency toward a copper content which corresponds to 1 atom of copper per oxime function.

TABLE 1

| | | | Number of moles | | |
|---|---|---|---|---|---|
| | Number of moles | Number of moles | of complexed | Theoretical | Oxime/Cu⁺⁺ |
| | of oxime | of Cu⁺⁺ | Cu⁺⁺ | oxime/Cu⁺⁺ | complexation |
| pH | (mol) | introduced (mol) | (potentiometry) | complexation ratio | ratio obtained |
| pH 3 | 3.73 10⁻⁴ | 4.658 10⁻⁵ | 3.33 10⁻⁵ | 8 | 11.2 |
| ″ | 3.73 10⁻⁴ | 9.37 10⁻⁵ | 5.25 10⁻⁵ | 4 | 7.1 |
| ″ | 3.73 10⁻⁴ | 1.875 10⁻⁴ | 8.33 10⁻⁵ | 2 | 4.5 |
| ″ | 3.73 10⁻⁴ | 3.75 10⁻⁴ | 1.13 10⁻⁴ | 1 | 3.3 |
| pH 5 | 3.73 10⁻⁴ | 4.685 10⁻⁵ | 4.52 10⁻⁵ | 8 | 8.25 |
| | 3.73 10⁻⁴ | 9.37 10⁻⁵ | 8.45 10⁻⁵ | 4 | 4.4 |
| | 3.73 10⁻⁴ | 1.875 10⁻⁴ | 1.50 10⁻⁴ | 2 | 2.5 |
| | 3.73 10⁻⁴ | 3.75 10⁻⁴ | 2.33 10⁻⁴ | 1 | 1.6 |
| | 1.80 10⁻⁶ | 3.75 10⁻⁴ | 1.49 10⁻⁴ | 0.48 | 1.2 |
| | 9.00 10⁻⁵ | 3.75 10⁻⁴ | 3.67 10⁻⁵ | 0.24 | 2.45 |

The complexes which are formed are stable since there is no variation in the quantity of copper ions when supernatants are placed at either pH 3 or pH 5 for 50 hours.

The complexation pH range is between 2.5 and 5.5.

Finally, when there is a precipitate, this precipitate is indeed a complex since a shoulder at 1525 cm⁻¹ and a displacement of the band at 1613 cm⁻¹ are observed when compared with an infrared spectrum of the reference oximated guar.

Complexation of the Oxime-Modified Guar in the Presence of Uranium:

The assays were carried out, at a constant pH (pH=3), by adding varying quantities of uranyl nitrate to a solution of oximated guar.

The concentration of the polymer is 16.8 g/l and the pH is adjusted by adding hydrochloric acid. The solution is then left to stir at ambient temperature for 24 hours.

The appearance of an intense yellow color and the formation of a precipitate are observed (see table 2).

The precipitate is dissolved in 37% hydrochloric acid (destruction of the complex and degradation of the polymer) and the medium is then directly analyzed by ICP-AES (inductively coupled plasma-atomic emission spectrometry).

Assaying the uranium ions confirms the formation of an oximated guar/uranium complex.

TABLE 2

| | Number of | | Number of moles |
|---|---|---|---|
| Number of | UO₂++ moles | | of uranium in |
| oxime moles | introduced | | the precipitate |
| (mol) | (mol) | Color | (ICP) |
| 3.74 10⁻⁴ | 4.685 10⁻⁵ | intense | |
| | | yellow, | |
| | | soluble | |
| 3.74 10⁻⁴ | 9.37 10⁻⁵ | intense | |
| | | yellow, | |
| | | soluble | |
| 3.74 10⁻⁴ | 1.875 10⁻⁴ | intense | 2.7 10⁻⁵ |
| | | yellow, | |
| | | precipitate | |
| 3.74 10⁻⁴ | 3.75 10⁻⁴ | intense | 1.9 10⁻⁵ |
| | | yellow, | |
| | | precipitate | |

Complexation of the Amine-Modified Guar

In a mixed solution, a change in color, which is a sign of the formation of a complex, is observed.

### Example 5

### Synthesizing Guar Carrying Ketone Functions

A solution of oxidizing agent is prepared by dissolving 1.45 g of sodium bromide in 90 ml of water. The temperature of the solution is lowered by plunging it into an ice bath (0-4° C.) before adding 5 ml of a solution of sodium hydrochlorite which has been standardized to 1.39 M. The pH is adjusted to about 10 by adding hydrochloric acid (1 N). The solution is left to stir for one hour before introducing 5.8 mg of 4-methoxy-2,2,6,6-tetramethyl-1-piperidyloxy to it.

In addition, 1.5 g of guar (weight average molar mass: 50 000 g/mol—Meyprogat® 7, marketed by Rhodia Chimie) are dissolved in 30 ml of water This guar solution is added dropwise to the solution of oxidizing agent.

During the addition, and until the pH no longer changes, sodium hydroxide solution (0.5 N) is added so as to maintain the pH at about 10.

When the pH has stabilized, 3 ml of methanol are introduced and the reaction medium is allowed to return to ambient temperature before being neutralized to pH 7 by adding 1 N hydrochloric acid.

The polymer is recovered by pouring the reaction mixture into ethanol and filtering the resulting precipitate on a no. 4 frit.

The polymer is then dissolved in 50 ml of water and the solution is placed in a dialysis membrane for 3 days.

This solution is finally frozen in an acetone/dry ice mixture and lyophilized.

The resulting product can then be used as the subject of the step described in example 2.

1-25. (canceled)

26. A polymer derived from a copolymeric polysaccharide formed from a main chain comprising similar or different anhydrohexose units and branches which comprise at least one neutral or anionic anhydropentose and/or anhydrohexose unit; said derived polymer comprising one or more units carrying an oxime function at least in the C2 position and being capable of being obtained by implementing the following steps:
a) bringing a polysaccharide into contact with an aqueous solution which comprises at least one oxidizing agent which enables at least the hydroxyl radical carried by the C2 carbon of one or more units to be oxidized to a ketone function; and
b) bringing the resulting polymer into contact with hydroxylamine, or a derivative, in order to transform the ketone function into an oxime function.

27. The polymer as claimed in claim 26, wherein the oxime function is located on the C2 carbon of the unit, on the C3 carbon, or on the C4 carbon.

28. The polymer as claimed in claim 26, wherein one or more of the units carry(ies) a carboxylic acid function (—COOH) on the carbon atom in position C6.

29. The polymer as claimed in claim 26, wherein the polysaccharide is a galactomannan.

30. The polymer as claimed in claim 29, wherein the galactomannan is guar, carob, tara or cassia gum.

31. The polymer as claimed in claim 26, wherein the polysaccharide is used in the form of an aqueous solution.

32. The polymer as claimed in claim 26, wherein step a) is performed in homogeneous phase.

33. The polymer as claimed in claim 26, wherein the polysaccharide is used in the form of a powder or particles.

34. The polymer as claimed in claim 26, wherein the reaction is performed in heterogeneous phase.

35. The polymer as claimed in claim 26, wherein the oxidizing agent is a bromine or a periodate of an alkali metal.

36. The polymer as claimed in claim 26, wherein the oxidizing agent has a molar ratio relative to the functions to be oxidized of less than 6., more particularly less than 4 and preferably between 1 and 2.5.

37. The polymer as claimed in claim 36, wherein the molar ratio is between 1 and 2.5.

38. The polymer as claimed in claim 26, wherein step a) is performed by adding the oxidizing agent to the polysaccharide.

39. The polymer as claimed in claim 26, wherein step a) is performed while maintaining the pH of the aqueous solution at a value which is between 6 and 8.

40. The polymer as claimed in claim 26, wherein the pH is maintained at between 6 and 9.5 during step b).

41. The polymer as claimed in claim 26, wherein step b) is implemented in the presence of a molar ratio of hydroxylamine or derivative to the ketone functions to be transformed of between 1 and 10.

42. The polymer as claimed in claim 41, wherein step b) is implemented using an aqueous solution of hydroxylamine or derivative.

43. The polymer as claimed in claim 26, wherein the polymer resulting from step a) or the polymer resulting from step b) is recovered by being precipitated in a non-solvent of the polymer.

44. A polymer derived from a copolymeric polysaccharide formed from a main chain which comprises similar or different anhydrohexose units and branches which comprise at least one neutral or anionic anhydropentose and/or anhydrohexose unit, said polymer comprising one or more units which carry(ies) an amine function at least in the C2 position and being capable of being obtained by the step c) consisting in bringing the polymer possessing one or more units carrying an oxime function at least in the C2 position, as defined in claim 26, into contact with an agent which reduces the oxime function.

45. The polymer as claimed in claim 44, wherein the amine function is located on the C2 carbon, on the C3 carbon, or on the C4 carbon of the unit.

46. The polymer as claimed in claim 44, wherein one or more of the units carry(ies) a carboxylic acid function (—COOH) on the carbon atom in the C6 position.

47. The polymer as claimed in claim 44, wherein step c) is implemented in the presence of lithium aluminum hydride; or of boron compounds, optionally combined with a Lewis acid which is molybdenum oxide, nickel chloride, titanium chloride or titanium oxide.

48. The polymer as claimed in claim 44, wherein step c) is implemented at a pH of between 3 and 10.

49. The polymer as claimed in claim 44, wherein step c) is implemented under an inert atmosphere.

50. An agent for complexing cationic species, optionally metallic cationic species, comprising a polymer as defined in claim 26.

51. A retention agent in the papermaking industry, optionally mixed with a multivalent metal, comprising a polymer as defined in claim 26.
